(19) United States
(12) Patent Application Publication
(10) Pub. No.: US 2024/0241119 A1
(43) Pub. Date: Jul. 18, 2024

(54) PREDICTION OF STEM CELL THERAPY RESPONSIVENESS BY QUANTIFICATION OF PRE-EXISTING B REGULATORY CELLS

(71) Applicant: Therapeutic Solutions International, Inc., Oceanside, CA (US)

(72) Inventors: Thomas E. ICHIM, Oceanside, CA (US); James VELTMEYER, Oceanside, CA (US); Timothy G. DIXON, Oceanside, CA (US)

(73) Assignee: Therapeutic Solutions International, Inc., Oceanside, CA (US)

(21) Appl. No.: 18/410,531

(22) Filed: Jan. 11, 2024

Related U.S. Application Data
(60) Provisional application No. 63/439,327, filed on Jan. 17, 2023.

(51) Int. Cl. G01N 33/569

(52) U.S. Cl. CPC G01N 33/56972; G01N 2333/5428; G01N 2333/70596

(57) ABSTRACT

Disclosed are novel means of stratifying patients into potential of positive response to mesenchymal stem cell therapy based on quantification of pretreatment levels of B regulatory cells. In one embodiment quantification of cells concurrently expressing CD5 and CD19. In another embodiment B regulatory cells are CD19+CD39−IL10+. In one embodiment the selection of B regulatory cells is quantified by flow cytometric means and patients possessing more than 7% IL-10 secreting CD19 cells are chosen for stem cell therapy. In some embodiments numbers of B regulatory cells are increased prior to treatment by administration of various interventions including providing GM-CSF, microbiome alteration or manipulation of oxidative stress.

### CROSS-REFERENCE TO RELATED APPLICATIONS

This application claims the benefit of priority to U.S. Provisional Application No. 63/439,327, filed on Jan. 17, 2023, which is incorporated herein by reference in its entirety.

### BACKGROUND OF THE INVENTION

Stem cell therapeutics have historically been associated with a high degree of variability in clinical trials. Unfortunately, in many situations stem cell therapeutics appear highly promising in early phases of clinical trials, however when cells enter more advanced stages of clinical testing, such as Phase III, the advantage over placebo is many times reduced.

Mesenchymal stem cells (MSCs) are the formative pluripotential blast cells found inter alia in bone marrow, blood, dermis and periosteum that are capable of differentiating into more than one specific type of mesenchymal or connective tissue (i.e. the tissues of the body that support the specialized elements; e.g. adipose, osseous, stroma, cartilaginous, elastic and fibrous connective tissues) depending upon various influences from bioactive factors, such as cytokines. The potential to differentiate into cells such as osteoblasts and chondrocytes is retained after isolation and expansion in culture; differentiation occurs when the cells are induced in vitro under specific conditions or placed in vivo at the site of damaged tissue. Epitopes on the surface of the human mesenchymal stem cells (hMSCs) are reactive with certain monoclonal antibodies known as SH2, SH3 and SH4 described in U.S. Pat. No. 5,486,359. These antibodies can be used as reagents to screen and capture the mesenchymal stem cell population from a heterogeneous cell population, such as exists, for example, in bone marrow.

It is known that MSC are cells capable of differentiating into not only mesenchymal cells but also diverse cells beyond the germ layer and have an ability of controlling the tissue development, repair and reproduction. Since the isolation and cultivation of mesenchymal stem cells are easy and mesenchymal stem cells have a potent proliferation potential, it is possible to ensure the number of transplantable cells in a short period. In addition, mesenchymal stem cells can be transplanted autologously with no immunological rejection, which causes few ethical problems. Furthermore, allogenic transplantation of mesenchymal stem cells without pretreatment is realistic because of their low immunogenicity. Accordingly, the therapeutic applications of mesenchymal stem cells as a material for ideal cell transplant therapy for diverse diseases are proceeding. When used for disease therapy, it is necessary to prepare mesenchymal stem cells with a certain level of quality rapidly and in a large amount. In order to allow the mesenchymal stem cells obtained from donors to proliferate in vitro, growth promoting substances, cell culture substrates and the like are variously examined.

One reason for this may be that there are different cells that the stem cells use to mediate their therapeutic effects. The current invention describes novel ways of predicting responsiveness to stem cell therapy.

### BRIEF DESCRIPTION OF THE DRAWINGS

FIG. 1 is a bar graph showing % of CD19 cells based on B regulatory cells expression of IL-10 and CD5.

### DESCRIPTION OF THE INVENTION

The invention provides the identification and quantification of B regulatory cells as a means of assessing ability to respond to stem cell therapy. In one embodiment stem cells administered are mesenchymal stem cells and the patients treated are patients suffering from neurological conditions. In a specific embodiment the cells used are mesenchymal stem cell from the umbilical cord. In a more specific embodiment the cells are cells commonly referred to in the art as “JadiCells”.

In other embodiments, isolated from peripheral blood or bone marrow. “Isolated” as used herein signifies that the cells are placed into conditions other than their natural environment. The term “isolated” does not preclude the later use of these cells thereafter in combinations or mixtures with other cells. A method of preparing human marrow mesenchymal stem cell cultures has been described in U.S. Pat. No. 5,486,359. Several techniques are known to those of skill in the art for the rapid isolation of mesenchymal stem cells. Approaches to mesenchymal stem cell isolation include leucopheresis, density gradient fractionation, immunoselection and differential adhesion separation. The cells used for therapy may be maintained in culture media which can be a chemically defined serum free media or can be a “complete medium”, such as Dulbecco's Modified Eagles Medium Supplemented with 10% serum (DMEM). Suitable chemically defined serum free media are described in U.S. Ser. No. 08/464,599 and WO96/39487, and “complete media” are described in U.S. Pat. No. 5,486,359. Chemically Defined Medium comprises a minimum essential medium such as Iscove's Modified Dulbecco's Medium (IMDM) (Gibco), supplemented with human serum albumin, human Ex Cyte lipoprotein, transfernin, insuin, vitamins, essential and non essential amino acids, sodium pyruvate, glutamine and a mitogen. These media stimulate mesenchymal stem cell growth without differentiation. For therapeutic purposes, the cells used may be isolated from peripheral blood or bone marrow may further be culture-expanded. The cells may be expanded, before or after freezing thereof, The media described herein are also suitable for the culture expansion of the mesenchymal stem cells. The cells may further be purified. In a preferred embodiment, “purified” indicates that the cell population contains less than 5% impurities, impurities being for example, cells that are not CD45+. The purified cell population can later be used in combinations or mixtures as is appropriate. The present invention contemplates any suitable method of employing monoclonal antibodies to separate mesenchymal stem cells from other cells, e.g., recovered from bone marrow. Accordingly, included in the present invention is a method of producing a population of mesenchymal stem cells comprising the steps of providing a cell suspension of tissue containing mesenchymal stem cells; contacting the cell suspension with one or a combination of monoclonal antibodies which recognize an epitope on the mesenchymal stem cells; and separating and recovering from the cell suspension the cells bound by the monoclonal antibodies. The monoclonal antibodies may be linked to a solid-phase and utilized to capture mesenchymal stem cells from tissue samples. The bound cells may then be separated from the solid phase by known methods depending on the nature of the antibody and solid phase. Monoclonal based systems appropriate for preparing the desired cell population include magnetic bead/paramagnetic particle column utilizing antibodies for either positive or negative selection; separation based on biotin or streptavidin affinity; and high speed flow cytometric sorting of immunofluorescent-stained mesenchymal stem cells mixed in a suspension of other cells. Thus, the method of the present invention includes the isolation of a population of hMSCs and enhancement using monoclonal antibodies raised against surface antigens expressed by marrow-derived hMSCs, i.e. SH2, SH3 or SH4. Deposits of the cell line cultures identified as SH2, SH3 and SH4 are on deposit with the American Type Culture Collection, 10801 University Blvd. Manassas, Va. 20110-2209, and are assigned the ATCC accession numbers HB 10743, BH 10744 and HB 10745, respectively. These monoclonal antibodies provide effective probes which can be utilized for identifying, quantifying, and purifying mesenchymal stem cells, regardless of their source in the body. In one embodiment, the isolation of the cell population of the present invention may comprise utilizing a combination of one or more antibodies that recognize a known marker on mesenchymal stem cells as well as an antibody which recognizes CD45. One method for such preparation of the precursor cells of the present invention is to first select a population of cells expressing a marker identifying mesenchymal stem cells, for example, SH3 or SH2 by immunomagnetic selection of a low density human bone marrow cell sample. Alternatively, it is contemplated that the initial cell selection can be based on the CD45 marker and the cell population be further characterized using the hMSC monoclonal antibodies.

In another embodiment, it is contemplated that a cell population can be selected based on the CD14 marker. CD14 is a membrane protein that functions as a receptor for endotoxin (lipopolysaccharide, LPS) and is expressed strongly on the surface of monocytes, but not expressed by myeloid progenitors.

B regulatory cell quantification techniques are well known in the art and including cellular counting, microscopic counting, flow cytometry, mass cytometry and quantification by image analysis. In one embodiment the invention describes the quantification of B regulatory cells producing interleukin-10 as being associated with response to mesenchymal stem cell therapy. The present invention relates to a regulatory subset of the normal B cell population characterized phenotypically as CD1dhighCD5+, and functionally by its ability to produce IL-10. The invention also relates to therapeutic uses of this regulatory B cell population. The regulatory B cell phenotype can be determined by antibody staining and flow cytometry, FACS, using antibodies to CD1d and CD5 and techniques known in the art, including but not limited to those described in the examples, infra. See, e.g., Section 6 et seq. The invention is based, in part, on the surprising discovery that cellular compositions enriched by selection for both CD1dhigh and CD5 cellular markers will contain a high percentage of IL-10 producing B cells than a population enriched with only one of these markers. The ability of the cells to produce IL-10 can be assessed by measuring IL-10 production in naïve cells and in cultured cells stimulated with LPS (lipopolysaccharide), PMA (phorbol 12-myristate 13-acetate), ionomycin, CpG or comparable stimulatory Toll-like receptor agonists, or with an agonist of CD40 (e.g., using an antibody to CD40). Production of IL-10 by the cells can be assessed by assaying for IL-10 in the cell culture supernatant. In addition, production of IL-10 can be verified directly by intracellular cytokine staining. Standard immunoassays known in the art can be used for such purpose. Examples of assays for IL-10 production are described in Section 6, infra. While IL-10 is produced at low levels in the naïve CD1dhighCD5+ B cell subset, IL-10 production is increased in response to stimulation.

For the detection of stem cell responsiveness. The enriched, isolated and/or purified regulatory B cell subset composition can comprise anywhere from 0.5% to 50%, at least 55%, at least 60%, at least 65%, at least 70%, at least 75%, at least 80%, at least 85%, at least 90%, at least 95%, at least 99%, or 100% regulatory B cells having the CD1dhighCD5+ phenotype that produce IL-10 (as determined by the assays described above). In a preferred embodiment, the enriched/purified regulatory B cell subset comprises greater than 50% regulatory B cells having the CD1dhighCD5+ phenotype. In a more preferred embodiment, the enriched/purified regulatory B cell subset comprises greater than 75% regulatory B cells having the CD1dhighCD5+ phenotype. In a still more preferred embodiment, the enriched/purified regulatory B cell subset comprises greater than 90% regulatory B cells having the CD1dhighCD5+ phenotype. The enriched, isolated and/or purified CD1dhighCD5+ regulatory B cells can be obtained from a mammalian subject, including but not limited to rodents, e.g. mice, rats; livestock, e.g. pigs, horses, cows, etc., pets, e.g. dogs, cats; and primates, e.g. humans. In one embodiment, the subject is an animal model of an IL-10 associated disease. The phenotypic markers described herein were identified in murine models; however, the invention contemplates that the cognate human regulatory B cell population will also produce IL-10, will be phenotypically distinct from other B cell populations, and will likely utilize the same transcription factors and display the same cell surface markers. Alternatively, the regulatory B cells may be enriched/purified from any tissue where they reside including, but not limited to, blood (including blood collected by blood banks), spleen, bone marrow, tissues removed and/or exposed during surgical procedures, and tissues obtained via biopsy procedures. Tissues/organs from which the regulatory B cells are enriched, isolated, and/or purified may be isolated from both living and non-living subjects, wherein the non-living subjects are organ donors. Methods for the isolation of the regulatory B cells are based on selecting cells having the CD1dhighCD5+ cell-specific markers; however, additional markers can be included for selection, such as CD19high. In a particular aspect of this embodiment, a population of regulatory B cells is enriched/purified by flow cytometry as demonstrated in the examples described in Section 6, infra. However, a variety of cell separation techniques known in the art can be used, including but not limited to magnetic separation using antibody-coated magnetic beads and/or particles, FACS, affinity chromatography, affinity column separation, “panning” with antibody attached to a solid matrix, density gradient centrifugation, and counter-flow centrifugal elutriation. (See, e.g., Kumar and Lykke, 1984, Pathology, 1:53-62). Regulatory B cells can be enriched by selecting cells having the CD1dhighCD5+ surface markers and separating using automated cell sorting such as fluorescence-activated cell sorting (FACS), solid-phase magnetic beads, etc. as demonstrated in examples described in sections 6 and 7 infra. To enhance enrichment, positive selection may be combined with negative selection; i.e., by removing cells having surface markers specific to non-B cells and/or those specific to non-regulatory B cells. Non-limiting examples of methods of negative selection are described supra. Exemplary surface markers specific to non-regulatory B cells include CD3, CD4, CD7, CD8, CD15, CD16, CD34, CD56, CD57, CD64, CD94, CD116, CD134, CD157, CD163, CD208, F4/80, Gr-1, and TCR.

### Example 1

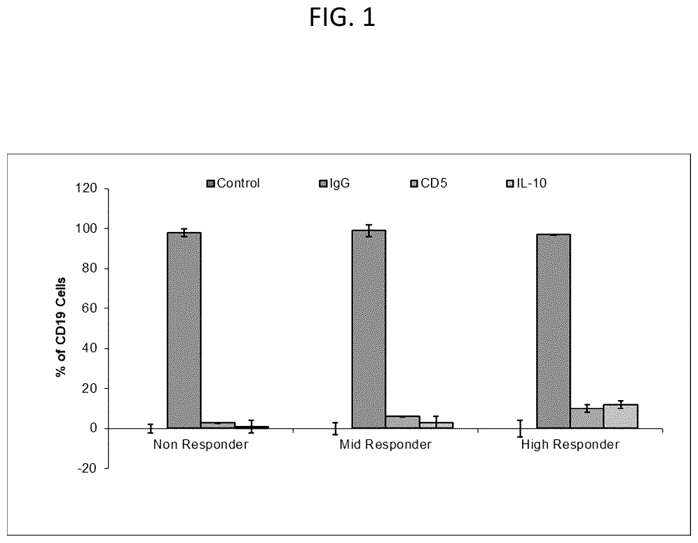

Patients receiving 200 million JadiCell mesenchymal stem cells for treatment of degenerative conditions were stratified into non-responders, medium responders and high responders based on physician evaluation and standardized quality of life scores. Cells were fractionated into peripheral blood mononuclear cells and analysis for co-expression of control and B regulatory cell numbers. B regulatory cells were identified as IL-10 expressing and/or CD5 expressing. Surface marker quantification was performed by the use of flow cytometry. Results are shown in FIG. 1.

1. A method of predicting response to mesenchymal stem cell therapy by quantification of B regulatory cells in a patient.

2. The method of claim 1, wherein said B regulatory cells express interleukin-10.

3. The method of claim 1, wherein said B regulatory cells are CD19+CD39−IL10+.

4. The method of claim 1, wherein said B regulatory cells are CD5 positive and CD19 positive.

5. The method of claim 1, wherein said B regulatory cells are IL-10 positive and CD19 positive.

6. The method of claim 1, wherein said B regulatory cells are FGF-1 positive.

7. The method of claim 1, wherein said B regulatory cells are FGF-2 positive.

8. The method of claim 1, wherein said B regulatory cells are FGF-5 positive.

9. The method of claim 1, wherein said B regulatory cells are IL-3 receptor positive.

10. The method of claim 1, wherein said B regulatory cells are IL-6 receptor positive.

11. The method of claim 1, wherein said B regulatory cells are capable of differentiating into monocytes.

12. The method of claim 1, wherein said B regulatory cells are capable of suppressing proliferation of an activated T cell.

13. The method of claim 1, wherein said B regulatory cells are capable of suppressing proliferation of an activated NKT cell.

14. The method of claim 1, wherein said B regulatory cells are capable of suppressing proliferation of an activated gamma delta T cell.

15. The method of claim 1, wherein said B regulatory cells are capable of suppressing proliferation of an activated T cell.

16. The method of claim 1, wherein said B regulatory cells are capable of inducing production of IL-10 in T cells.

17. The method of claim 1, wherein said B regulatory cells are capable of suppressing proliferation of an activated T cell.
